(19) United States
(12) Patent Application Publication
(10) Pub. No.: US 2025/0043239 A1
(43) Pub. Date: Feb. 6, 2025

(54) STREPTOMYCES GRAMINEARUS, STREPTOMYCES GRAMINEARUS OIL SUSPENSION, AND ITS PREPARATION METHOD AND APPLICATION THEREOF

(71) Applicants: Hubei Biopesticide Engineering Research Centre, Wuhan (CN); Hubei Kanwin Biotechnology Co., Ltd, Wuhan (CN)

(72) Inventors: Hongqu WU, Wuhan (CN); Fang LIU, Wuhan (CN); Shaoyong KE, Wuhan (CN); Zhigang ZHU, Wuhan (CN); Tianwu YANG, Wuhan (CN)

(73) Assignees: Hubei Biopesticide Engineering Research Centre, Wuhan (CN); Hubei Kanwin Biotechnology Co., Ltd, Wuhan (CN)

(21) Appl. No.: 18/789,990

(22) Filed: Jul. 31, 2024

(30) Foreign Application Priority Data
Jul. 31, 2023 (CN) 2023109514175

(51) Int. Cl. C12N 3/00; C12N 1/20; C12R 1/465

(52) U.S. Cl. CPC C12N 3/00; C12N 1/205; C12R 2001/465

(57) ABSTRACT

The invention provides a streptomyces graminearus strain, a streptomyces graminearus oil suspension, as well as its preparation method and application thereof. The streptomyces graminearus oil suspension contains streptomyces graminearus conidiospore powder, emulsifier, stabilizer, wetting dispersant, antioxidant, thickener, ultraviolet protectant, preservative and carrier oil. The use of carrier oil as the dispersion medium offers environmental friendliness, excellent permeability, adhesion properties, and resistance to rain washing. Additionally, the carrier oil enhances the spore germination of streptomyces graminearus. Furthermore, it has the advantages of low application cost, slow spore settling speed, easy to shake well when using, stable efficacy, long storage time, suitability for various spraying techniques and a simple preparation method. The spore survival time of the streptomyces graminearus oil suspension is prelonged, with a spore germination rate is as high as 85% after 18 months of storage. It demonstrates effective control against yam, peanut and forest nematode.

### CROSS-REFERENCE TO RELATED APPLICATIONS

The application claims priority to Chinese patent application No. 2023109514175, filed on Jul. 31, 2023, the entire contents of which are incorporated herein by reference.

### SEQUENCE LISTING

The sequence listing xml file submitted herewith, named “JZWH23426.xml”, created on May 24, 2023, and having a file size of 3,321 bytes, is incorporated by reference herein.

### TECHNICAL FIELD

The invention relates to the technical field of preparation and processing of microbial pesticides, in particular to a streptomyces graminearus strain, a streptomyces graminearus oil suspension, as well as its preparation method and application thereof.

### BACKGROUND

Plant nematode disease causes global crop losses of at least $157 billion annually, and is about to become the world's primary crop disease. In China, the nematode disease results in all kinds of agricultural crop annual average yield loss of 10%-15%. In addition, the serious occurrence of pine wood nematode disease not only poses a great threat to the pine forest ecosystem in China, but also causes economic losses of several billion yuan every year.

At present, chemical control methods for plant nematodes are mainly used worldwide, such as soil fumigation and disinfection (such as Mianlong, chloropicrin, and metham-sodium, etc.), and chemical nematicides (such as fosthiazate, carbosulfan, etc.). The chemical control methods can better inhibit the occurrence of the nematode disease and reduce the loss of agriculture and forestry; However, the extensive use of the chemical pesticides not only causes environmental pollution, but also causes serious soil degradation, resulting in crop production reduction year by year. In order to solve the above problems, it is imperative to develop efficient and environmentally friendly biological nematicides.

Actinomycetes are ubiquitous in soil, with wide variety species and large quantities, and in recent years, they are considered as a potential research area of secondary metabolites with broad prospects. According to incomplete statistics, 8,600 active metabolites have been isolated from actinomycetes, with multiple structural types. From just over 10 compounds in 1940 to more than 22,000 compounds in 2002, the number of active metabolites has increased exponentially during this period, and the proportion of secondary metabolites produced by actinomycetes has an expanding trend in recent years. As a kind of actinomyces, Streptomyces metabolites have novel chemical structures, unique target, broad spectrum, low toxicity, low residue, and are less affected by the environment, etc. Therefore, the development of live Streptomyces pesticides formulations is significant for both the ecological environment and socioeconomic development.

However, there is currently no technical proposal for using Streptomyces to prepare nematicides.

### SUMMARY

In view of the above, the invention provides a streptomyces graminearus strain, a streptomyces graminearus oil suspension, as well as its preparation method and application thereof, so as to solve or at least partially solve the technical problems existing in the prior art.

In the first aspect, the invention provides a streptomyces graminearus strain, the taxonomic name of the strain is streptomyces graminearus NBERC-VWS578, and the strain has a deposit accession number of CCTCC NO: M 2022512.

In the second aspect, the invention also provides a streptomyces graminearus conidiospore powder prepared by activating and culturing the streptomyces graminearus strain to produce spores, thereby obtaining the aforementioned streptomyces graminearus conidiospore powder.

Preferably, a preparation method of the aforementioned streptomyces graminearus conidiospore powder comprises the following steps:
Inoculating the streptomyces graminearus strain onto a PDA medium plate, activating and culturing until sporulation, and collecting spores powder from the PDA medium plate culture colony to prepare a suspension with a concentration of (2-4)×10⁷ spores/mL;
Adding 0.5-2 mL of the suspension to a PD culture medium to achieve a spore concentration of (0.5-2)×10⁶ spores/mL, shaking culture, then transfering into a PDA culture medium, and continuing shaking culture to obtain an inoculum;
Adding rice to the inoculum, and culturing for sporulation to obtain a rice culture;
After freezing and vacuum drying the rice culture, separating the spores powder from the rice substrate, thereby obtaining the streptomyces graminearus conidiospore powder.

In the third aspect, the invention also provides a streptomyces graminearus oil suspension, which contains the aforementioned streptomyces graminearus conidiospore powder.

Preferably, the streptomyces graminearus oil suspension contains the following raw materials by mass fraction: 3-25% of the streptomyces graminearus conidiospore powder, 5-10% of emulsifier, 0.5-3% of stabilizer, 3-8% of wetting dispersant, 0.1-3% of antioxidant, 3-8% of thickener, 0.1-0.5% of ultraviolet protectant, 0.1-3% of preservative and 39.5-85.2% of carrier oil.

Preferably, in the streptomyces graminearus oil suspension, the carrier oil comprises at least one of rosin-based vegetable oil, soybean oil, sunflower seed oil, and corn oil;
and/or, the emulsifier comprises at least one of SP-OF3472B, DR7792, SP-OF3477, TERMUL2500, TERMUL3015, LT-F192, and op-10;
and/or, the stabilizer comprises at least one of CMC-Na, BHA, and epoxidized soybean oil;
and/or, the wetting dispersant comprises at least one of Morwet EFW, TERWET 1010, MF-5, Morwet D425, calcium lignosulphonate, TERSPERSE 2020, and NNO;
and/or, the antioxidant comprises at least one of vitamin C, vitamin E, and BHA;
and/or, the thickener comprises at least one of silica (white carbon black) and organic bentonite;
and/or, the ultraviolet protectant comprises at least one of dextrin, xanthan gum, sodium fluorescein, vitamin C, and nano zinc oxide;
and/or, the preservative comprises at least one of sodium diacetate and calcium propionate.

Preferably, the streptomyces graminearus conidiospore powder has a spore content greater or equal to 7×10⁹ spores/g, and a moisture content of 2-5% by weight.

In the fourth aspect, the invention also provides a method for preparing the aforementioned streptomyces graminearus oil suspension, comprising the following steps:

Mixing the streptomyces graminearus conidiospore powder, the emulsifier, the stabilizer, the wetting dispersant, the antioxidant, thickener, the ultraviolet protectant, the preservative, and the carrier oil, and stirring to obtain the streptomyces graminearus oil suspension.

Preferably, the method for preparing the streptomyces graminearus oil suspension comprises the following steps:
Mixing the emulsifier, the stabilizer, the wetting dispersant, the antioxidant, the thickener, the ultraviolet protectant, the preservative, and the carrier oil, and shearing at 8000-10000 r/min to obtain a mixture;
Mixing the mixture with the streptomyces graminearus conidiospore powder, and grinding to obtain the streptomyces graminearus oil suspension.

In the fifth aspect, the invention also provides the aforementioned streptomyces graminearus conidiospore powder, the aforementioned streptomyces graminearus oil suspension, or the aforementioned method for preparing the streptomyces graminearus oil suspension in preparing nematicides.

Compared with the prior art, the streptomyces graminearus strain, the streptomyces graminearus oil suspension and its preparation method and application of the invention have the following beneficial effects:
the streptomyces graminearus oil suspension of the present invention contains streptomyces graminearus conidiospore powder, emulsifier, stabilizer, wetting dispersant, antioxidant, thickener, ultraviolet protectant, preservative and carrier oil. The use of carrier oil as the dispersion medium offers environmental friendliness, excellent permeability, adhesion properties, and resistance to rain washing. Additionally, the carrier oil enhances the spore germination of streptomyces graminearus. Furthermore, it has the advantages of low application cost, slow spore settling speed, easy to shake well when using, stable efficacy, long storage time, suitability for various spraying techniques and a simple preparation method. The spore survival time of the streptomyces graminearus oil suspension is prelonged, with a spore germination rate is as high as 85% after 18 months of storage. It demonstrates effective control against yam, peanut and forest nematode, etc, examples include yam root knot nematode, pratylenchus coffeae, peanut root knot nematode, tomato meloidogyne incongnita, bursaphelenchus xylophilus, etc.

### BRIEF DESCRIPTION OF DRAWINGS

In order to more clearly state the technical scheme in the embodiment of the invention or prior art, a brief introduction of the drawings needed in the description of the embodiment or prior art is given below. Obviously, the drawings described below are only some embodiments of the invention, and other drawings may be obtained from them without creative effort for ordinary people skilled in the art.

FIG. 1 shows a graph of streptomycesgraminearus NBERC-VWS578 under an optical microscope (1000×);

FIG. 2 shows a front plate graph of streptomyces graminearus NBERC-VWS578 on ISP2 (streptomyces graminearus culture medium No. 2);

FIG. 3 shows a reverse plate graph of streptomyces graminearus NBERC-VWS578 on ISP2 (streptomyces graminearus culture medium No. 2);

FIG. 4 shows a front plate graph of streptomyces graminearus NBERC-VWS578 on Gao's No. 1 culture medium;

FIG. 5 shows a reverse plate graph of streptomyces graminearus NBERC-VWS578 on Gao's No. 1 culture medium;

FIG. 6 shows the phylogenetic tree of streptomyces graminearus NBERC-VWS578.

### DETAILED DESCRIPTION OF THE EMBODIMENTS

The following is a clear and complete description of the technical scheme in combination with the embodiments of the invention. Obviously, the embodiments described below are only part of the embodiments of the invention, not all embodiments. Based on the embodiments of the invention, all other embodiments obtained by ordinary people skilled in the art without creative effort fall within the scope of protection of the invention.

It should be noted that the order of description of the following embodiments is not intended to qualify the preferred order of embodiments. In addition, in the description of this invention, the term “including” means “including but not limited to”. Various embodiments of this invention may exist in a range of forms; It should be understood that the range description is only convenient and concise, and should not be construed as a hard limit on the scope of protection of the invention; Therefore, the range description should be considered to have specifically disclosed all possible subranges and a single numerical value within that range. For example, a range description from 1 to 6 should be considered to have specifically disclosed subranges, such as from 1 to 3, from 1 to 4, from 1 to 5, from 2 to 4, from 2 to 6, from 3 to 6, etc., as well as single numbers within the range, such as 1, 2, 3, 4, 5, and 6, which are applicable regardless of the range. In addition, whenever a numerical range is indicated herein, it means a number (fraction or integer) including any reference within the indicated range.

In one embodiment, the invention provides a streptomyces graminearus strain, taxonomically classified as: streptomyces graminearus NBERC-VWS578, and the streptomyces graminearus strain has a deposit number of CCTCC NO: M 202251, which was deposited on May 5, 2022 at China Center for Type Culture Collection in Wuhan University, Wuhan, China.

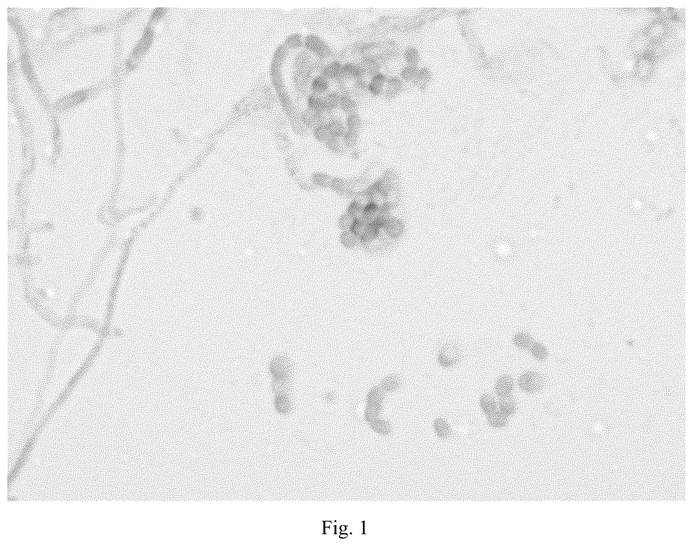

Specifically, a graph of the streptomyces graminearus NBERC-VWS578 under an optical microscope (1000×) is shown in FIG. 1.

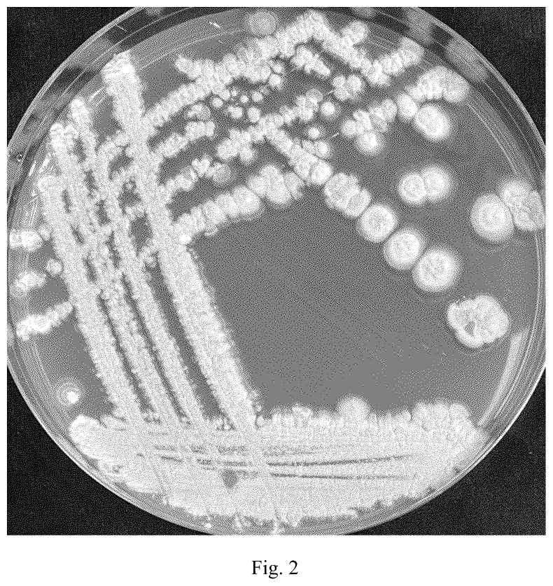

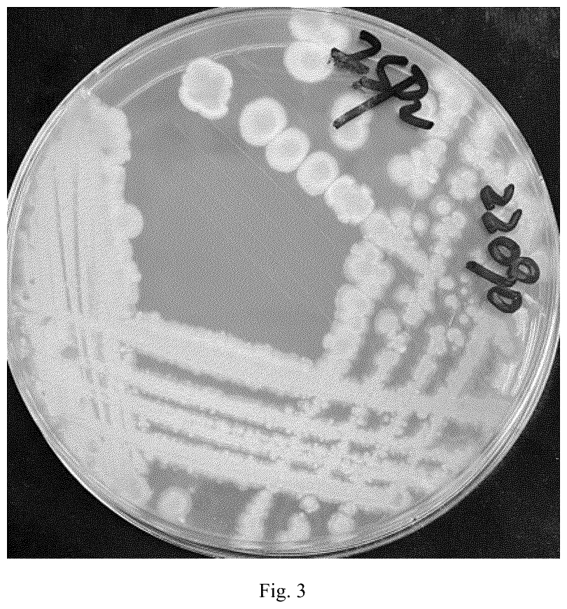

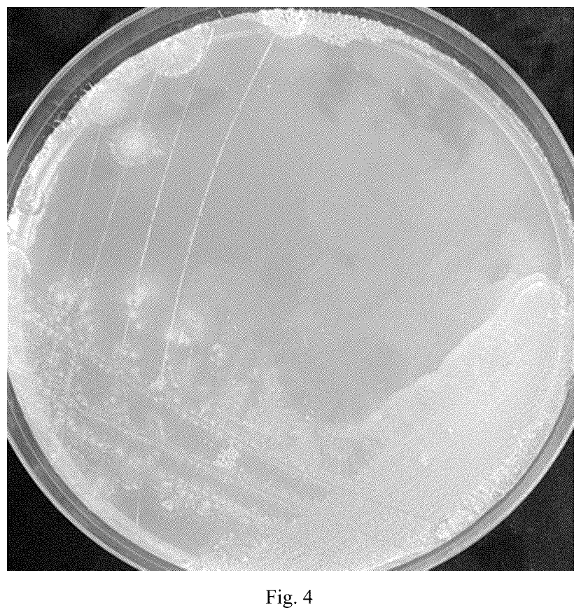

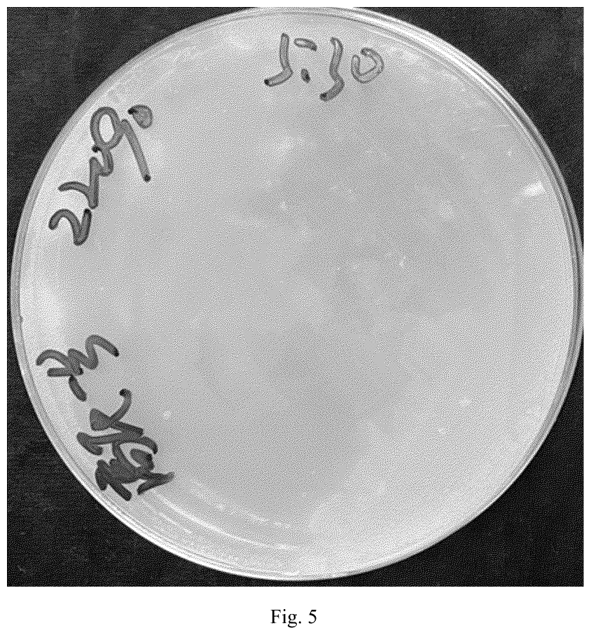

The front plate graph of the streptomyces graminearus NBERC-VWS578 strain on ISP2 (streptomyces graminearus culture medium No. 2) is shown in FIG. 2. The reverse plate graph of the streptomyces graminearus NBERC-VWS578 strain on ISP2 (streptomyces graminearus culture medium No. 2) is shown in FIG. 3. The front plate graph of the streptomyces graminearus NBERC-VWS578 strain on Gao's No. 1 culture medium is shown in FIG. 4. The reverse plate graph of the streptomyces graminearus NBERC-VWS578 strain on Gao's No. 1 culture medium is shown in FIG. 5.

The morphological characteristics of the streptomyces graminearus NBERC-VWS578 strain are as follows: the strain grows generally on the Gao's No. 1 culture medium, with gray-white colonies and without soluble pigment, the colonies exhibit concentric rings in the center and irregular edges. Under an optical microscope, the spore chains are curled, and spores are spherical or near-elliptical.

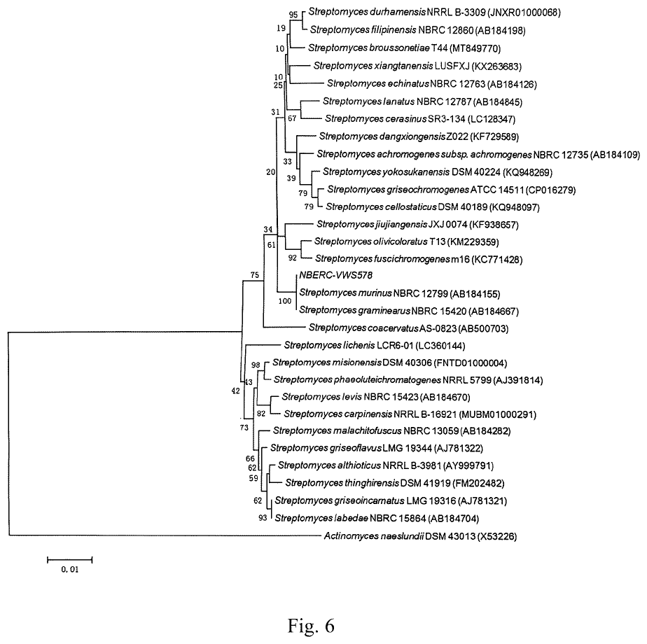

Based on 16S rRNA gene sequence comparison, the phylogenetic tree of the streptomyces graminearus NBERC-VWS578 strain constructed by neighbor-joining method is shown in FIG. 6, with Actinomyces naeslundii DSM 43013 (X53226) as the outer branch.

The 16S rRNA gene sequence of the streptomyces graminearus NBERC-VWS578 strain is shown in the sequence listing.

(SEQ ID NO: 1)
CATGCAAGTCGAACGATGAAGCCCTTCGGGGTGGATTAGTGGCGAACGGG
TGAGTAACACGTGGGCAATCTGCCCTGCACTCTGGGACAAGCCCTGGAAA
CGGGGTCTAATACCGGATATGACCATCTTGGGCATCCTTGATGGTGTAAA
GCTCCGGCGGTGCAGGATGAGCCCGCGGCCTATCAGCTTGTTGGTGAGGT
AATGGCTCACCAAGGCGACGACGGGTAGCCGGCCTGAGAGGGCGACCGGC
CACACTGGGACTGAGACACGGCCCAGACTCCTACGGGAGGCAGCAGTGGG
GAATATTGCACAATGGGCGAAAGCCTGATGCAGCGACGCCGCGTGAGGGA
TGACGGCCTTCGGGTTGTAAACCTCTTTCAGCAGGGAAGAAGCGAAAGTG
ACGGTACCTGCAGAAGAAGCGCCGGCTAACTACGTGCCAGCAGCCGCGGT
AATACGTAGGGCGCAAGCGTTGTCCGGAATTATTGGGCGTAAAGAGCTCG
TAGGCGGCTTGTCACGTCGATTGTGAAAGCTCGGGGCTTAACCCCGAGTC
TGCAGTCGATACGGGCTAGCTAGAGTGTGGTAGGGGAGATCGGAATTCCT
GGTGTAGCGGTGAAATGCGCAGATATCAGGAGGAACACCGGTGGCGAAGG
CGGATCTCTGGGCCATTACTGACGCTGAGGAGCGAAAGCGTGGGGAGCGA
ACAGGATTAGATACCCTGGTAGTCCACGCCGTAAACGGTGGGAACTAGGT
GTTGGCGACATTCCACGTCGTCGGTGCCGCAGCTAACGCATTAAGTTCCC
CGCCTGGGGAGTACGGCCGCAAGGCTAAAACTCAAAGGAATTGACGGGGG
CCCGCACAAGCGGCGGAGCATGTGGCTTAATTCGACGCAACGCGAAGAAC
CTTACCAAGGCTTGACATACACCGGAAAGCATTAGAGATAGTGCCCCCCT
TGTGGTCGGTGTACAGGTGGTGCATGGCTGTCGTCAGCTCGTGTCGTGAG
ATGTTGGGTTAAGTCCCGCAACGAGCGCAACCCTTGTCCCGTGTTGCCAG
CAGGCCCTTGTGGTGCTGGGGACTCACGGGAGACCGCCGGGGTCAACTCG
GAGGAAGGTGGGGACGACGTCAAGTCATCATGCCCCTTATGTCTTGGGCT
GCACACGTGCTACAATGGCCGGTACAATGAGCTGCGATACCGTGAGGTGG
AGCGAATCTCAAAAAGCCGGTCTCAGTTCGGATTGGGGTCTGCAACTCGA
CCCCATGAAGTCGGAGTCGCTAGTAATCGCAGATCAGCATTGCTGCGGTG
AATACGTTCCCGGGCCTTGTACACACCGCCCGTCACGTCACGAAAGTCGG
TAACACCCGAAGCCGGTGGCCCAACCCCTTGTGGGAGGGAGCTGTCGAAG
GTGGGACCAGCGAT.

The 16S rRNA gene sequence BLAST results of the streptomyces graminearus NBERC-VWS578 strain are shown in Table 1 below.

TABLE 1
The 16S rRNA gene sequence BLAST results of the streptomyces graminearus NBERC-VWS578 strain

| | | | | Pairwise | Mismatch/ |
|---|---|---|---|---|---|
| Rank | Name | Strain | Accession | Similarity(%) | Total nt |
| 1 | Streptomyces murinus | NBRC 12799 | AB184155 | 100 | 0/1414 |
| 2 | streptomyces graminearus | NBRC 15420 | AB184667 | 100 | 0/1414 |
| 3 | Streptomyces xiangtanensis | LUSFXJ | KX263683 | 98.77786 | 17/1391 |
| 4 | Streptomyces misionensis | DSM 40306 | FNTD01000004 | 98.72702 | 18/1414 |
| 5 | Streptomyces phaeoluteichromatogenes | NRRL 5799 | AJ391814 | 98.58557 | 20/1414 |
| 6 | Streptomyces dangxiongensis | Z022 | KF729589 | 98.44413 | 22/1414 |
| 7 | Streptomyces broussonetiae | T44 | MT849770 | 98.44413 | 22/1414 |
| 8 | Streptomyces lanatus | NBRC 12787 | AB184845 | 98.16124 | 26/1414 |
| 9 | Streptomyces olivicoloratus | T13 | KM229359 | 98.16124 | 26/1414 |
| 10 | Streptomyces fuscichromogenes | m16 | KC771428 | 98.16124 | 26/1414 |
| 11 | Streptomyces durhamensis | NRRL B-3309 | JNXR01000068 | 98.09052 | 27/1414 |
| 12 | Streptomyces cerasinus | SR3-134 | LC128347 | 98.09052 | 27/1414 |
| 13 | Streptomyces filipinensis | NBRC 12860 | AB184198 | 98.09052 | 27/1414 |
| 14 | Streptomyces echinatus | NBRC 12763 | AB184126 | 98.09052 | 27/1414 |
| 15 | Streptomyces althioticus | NRRL B-3981 | AY999791 | 98.08102 | 27/1407 |
| 16 | Streptomyces malachitofuscus | NBRC 13059 | AB184282 | 98.0198 | 28/1414 |
| 17 | Streptomyces lichenis | LCR6-01 | LC360144 | 98.0198 | 28/1414 |
| 18 | Streptomyces griseochromogenes | ATCC 14511 | CP016279 | 97.94908 | 29/1414 |
| 19 | Streptomyces cellostaticus | DSM 40189 | KQ948097 | 97.94908 | 29/1414 |
| 20 | Streptomyces griseoincarnatus | LMG 19316 | AJ781321 | 97.87836 | 30/1414 |
| 21 | Streptomyces yokosukanensis | DSM 40224 | KQ948269 | 97.87836 | 30/1414 |

The physiological and biochemical characteristics and carbon source utilization of the streptomyces graminearus NBERC-VWS578 strain are shown in Table 2 below.

TABLE 2
The physiological and biochemical characteristics
and carbon source utilization of the streptomyces graminearus
NBERC-VWS578 strain

| Test item | | Result |
|---|---|---|
| A1 | negative control | − |
| A2 | dextrin | W |
| A3 | D-maltose | + |
| A4 | D-trehalose | + |
| A5 | D-cellobiose | + |
| A6 | gentiobiose | + |
| A7 | trehalulose | + |
| A8 | D-turanose | W |
| A9 | stachyose | W |
| A10 | positive control | + |
| A11 | pH 6 | + |
| A12 | pH 5 | + |
| B1 | raffinose | W |
| B2 | α-D-lactose | − |
| B3 | melibiose | W |
| B4 | β-formyl-D-glucoside | + |
| B5 | D-salicin | + |
| B6 | N-acetyl-D-glucosamine | + |
| B7 | N-acetyl-beta-D-mannosamine | − |
| B8 | N-acetyl-D-galactosamine | − |
| B9 | N-acetylneuraminic acid | − |
| B10 | 1% NaCl | + |
| B11 | 4% NaCl | + |
| B12 | 8% NaCl | + |
| C1 | α-D-glucose | + |
| C2 | D-mannose | + |
| C3 | D-fructose | + |
| C4 | D-galactose | W |
| C5 | 3-formylglucose | − |
| C6 | D-fucose | − |
| C7 | L-fucose | − |
| C8 | L-rhamnose | W |
| C9 | inosine | W |
| C10 | sodium lactate | + |
| C11 | fusidic acid | − |
| C12 | D-serine | − |
| D1 | D-sorbitol | + |
| D2 | D-mannitol | + |
| D3 | D-Arabinol | − |
| D4 | inositol | + |
| D5 | glycerol | + |
| D6 | D-glucose-6-phosphate | − |
| D7 | D-fructose-6-phosphate | − |
| D8 | D-aspartate | − |
| D9 | D-serine | − |
| D10 | troleandomycin | − |
| D11 | rifamycin SV | − |
| D12 | minocycline | − |
| E1 | gelatin | W |
| E2 | e aminoacetyl L-proline | − |
| E3 | L-alanine | + |
| E4 | L-arginine | W |
| E5 | L-aspartic acid | W |
| E6 | L-glutamic acid | W |
| E7 | L-histamine | + |
| E8 | L-pyroglutamic acid | W |
| E9 | L-serine | + |
| E10 | lincomycin | − |
| E11 | guanidine hydrochloride | − |
| E12 | tetradecyl sodium sulfate | − |
| F1 | pectin | W |
| F2 | D-galacturonic acid | W |
| F3 | L-galacturolactone | W |
| F4 | D-gluconic acid | W |
| F5 | D-glucuronic acid | − |
| F6 | glucuronamide | − |
| F7 | mucic acid | + |
| F8 | quinic acid | − |
| F9 | saccharic acid | + |
| F10 | vancomycin | − |
| F11 | tetrazole violet | − |
| F12 | tetrazole blue | − |
| G1 | p-hydroxyphenylacetic acid | − |
| G2 | methyl pyruvate | − |
| G3 | D-methyl lactate | − |
| G4 | L-lactic acid | + |
| G5 | citric acid | + |
| G6 | α-ketoglutaric acid | − |
| G7 | D-malic acid | − |
| G8 | L-malic acid | + |
| G9 | bromosuccinic acid | − |
| G10 | nalidixic acid | + |
| G11 | lithium chloride | + |
| G12 | potassium tellurite | + |
| H1 | Tween 40 | − |
| H2 | γ-aminobutyric acid | − |
| H3 | α-hydroxy-butyric acid | − |
| H4 | β-hydroxy-D,L-butyric acid | − |
| H5 | α-ketobutyric acid | − |
| H6 | acetoacetic acid | W |
| H7 | propionic acid | − |
| H8 | acetic acid | − |
| H9 | formic acid | W |
| H10 | aztreonam | + |
| H11 | sodium butyrate | + |
| H12 | sodium bromate | W |

In Table 2 above, +: positive reaction; −: negative reaction; W: weak positive reaction;

The above results identify the NBERC-VWS578 strain as: streptomyces graminearus.

In another embodiment, the invention provides a streptomyces graminearus conidiospore powder, a preparation method of the aforementioned streptomyces graminearus conidiospore powder is as follows: activating and culturing the streptomyces graminearus strain to produce spores, thereby obtaining the streptomyces graminearus conidiospore powder.

In some aspects, the preparation method of the streptomyces graminearus conidiospore powder comprises the following steps:
S1. Inoculating the streptomyces graminearus strain onto a PDA medium plate, activating and culturing until sporulation, and collecting spores powder from the PDA medium plate culture colony to prepare a suspension with a concentration of (2-4)×10⁷ spores/mL;
S2. Adding 0.5-2 mL of the suspension to a PD culture medium to achieve a spore concentration of (0.5-2)×10⁶ spores/mL, shaking culture, then transfering into a PDA culture medium, and continuing shaking culture to obtain an inoculum;
S3. Adding rice to the inoculum, and culturing for sporulation to obtain a rice culture;
S4. After freezing and vacuum drying the rice culture, separating the spores powder from the rice substrate, thereby obtaining the streptomyces graminearus conidiospore powder.

Specifically, the preparation method of the streptomyces graminearus conidiospore powder comprises the following steps:
S1. Inoculating the streptomyces graminearus strain onto a PDA medium plate, activating and culturing at 28° C. and 12L:12D (12 Light:12 Dark) until sporulation, and collecting spores powder from the PDA medium plate culture colony to prepare the suspension with a concentration of (2-4)×10⁷ spores/mL;
S2. Adding 0.5-2 mL of the suspension to a PD culture medium to achieve a spore concentration of (0.5-2)×10⁶ spores/mL, shaking culture at 28±1° C. and 180 r/min for 48 h, then transfering into 1000 mL of a PDA culture medium (PDA is standard fungal culture solution), and continuing shaking culture for 24 h, thus obtaining the inoculum;
S3. Placing 100 g of rice in a beaker, adding water according to the ratio of rice (g):water (mL)=1:1.5 (that is, adding 1.5 mL of water to each g of rice), soaking for 30 min, then washing and draining, and sterilizing for 30 min under the sterilization conditions of 121° C. and 0.1 MPa; After cooling, mixing the inoculum in step S2 with the rice according to the inoculation amount of 10% by volume, and the inoculated rice is poured into a Petri dish with a diameter of 15 cm and covered with plastic wrap; Culturing the inoculated at 28° C. and 12L:12D (12 Light:12 Dark) for sporulation, then removing the plastic wrap and culturing for 3-5 d to obtain the rice culture;
S4. After freezing and vacuum drying the rice culture, reducing the moisture rate to less than 4%, separating the spores powder from the rice substrate by vibrating screen, thereby obtaining the aforementioned streptomyces graminearus conidiospore powder. The spore content and viable spore rate were measured, the streptomyces graminearus conidiospore powder was stored in a refrigerator at 4° C. for later use.

Among them, PDA medium is potato glucose agar medium, PD culture medium is PD liquid medium, the PDA medium and PD culture medium used in this application are both commercially available medium/culture medium; 12L:12D refers to photoperiod, 12 hours of light, then 12 hours of dark.

In another embodiment, the invention provides a streptomyces graminearus oil suspension, which contains the aforementioned streptomyces graminearus conidiospore powder.

In some aspects, the streptomyces graminearus oil suspension contains the following raw materials by mass fraction: 3-25% of the streptomyces graminearus conidiospore powder, 5-10% of emulsifier, 0.5-3% of stabilizer, 3-8% of wetting dispersant, 0.1-3% of antioxidant, 3-8% of thickener, 0.1-0.5% of ultraviolet protectant, 0.1-3% of preservative and 39.5-85.2% of carrier oil.

In some aspects, the carrier oil comprises at least one of rosin-based vegetable oil, soybean oil, sunflower seed oil, and corn oil.

In some aspects, the emulsifier comprises at least one of SP-OF3472B (produced by Nanjing Qingyu Chemical Research Co., Ltd., china), DR7792, SP-OF3477, TERMUL2500, TERMUL3015, LT-F192 (LT-F192 emulsifier, produced by Shenzhen langtai biology science and technology co., Ltd, china), and op-10.

In some aspects, the stabilizer comprises at least one of CMC-Na (sodium carboxymethylcellulose), BHA (butyl hydroxyanisole), and epoxidized soybean oil.

In some aspects, the wetting dispersant comprises at least one of Morwet EFW (Akzonobel Morwet series dispersants), TERWET 1010, MF-5, Morwet D425 (Akzonobel dispersants), calcium lignosulphonate, TERSPERSE 2020 (produced by HUNTSMAN company Huntsman, American, alkyl naphthalene sulfonates), and NNO (2-naphthalenesulfonic acid formaldehyde polymer sodium salt).

In some aspects, the antioxidant comprises at least one of vitamin C, vitamin E, and BHA.

In some aspects, the thickener comprises at least one of silica (white carbon black) and organic bentonite.

In some aspects, the ultraviolet protectant comprises at least one of dextrin, xanthan gum, sodium fluorescein, vitamin C, and nano zinc oxide.

In some aspects, the preservative comprises at least one of sodium diacetate and calcium propionate.

In some aspects, the streptomyces graminearus conidiospore powder has a spore content greater or equal to 7×10⁹ spores/g, and a moisture content of 2-5% by weight.

Preferably, the streptomyces graminearus oil suspension contains the following raw materials by mass fraction: 5% of the streptomyces graminearus conidiospore powder, 8% of the SP-OF3472B, 0.5% of the epoxidized soybean oil, 10% of the Morwet EFW and calcium lignosulphonate, 0.3% of the vitamin C, 3% of the organic bentonite, 0.4% of the nano zinc oxide, 0.5% of the calcium propionate and 72.3% of the rosin-based vegetable oil (can be OD-1 rosin-based vegetable oils produced by Fujian Nord Biotechnology Co., LTD, china).

Preferably, the streptomyces graminearus oil suspension contains the following raw materials by mass fraction: 15% of the streptomyces graminearus conidiospore powder, 17% of the DR7792 (produced by Shenzhen langtai biology science and technology co., Ltd, china), 1.2% of the VE, 10% of the Morwet D425, 0.3% of the vitamin C, 8% of the white carbon black, 0.4% of the vitamin C, 0.5% of the calcium propionate and 47.6% of the corn oil.

In another embodiment, the invention provides a method for preparing the aforementioned streptomyces graminearus oil suspension, comprising the following steps:

Mixing the streptomyces graminearus conidiospore powder, the emulsifier, the stabilizer, the wetting dispersant, the antioxidant, the thickener, the ultraviolet protectant, the preservative, and the carrier oil, and stirring to obtain the streptomyces graminearus oil suspension.

In some aspects, mixing the emulsifier, the stabilizer, the wetting dispersant, the antioxidant, the thickener, the ultraviolet protectant, the preservative, and the carrier oil, and shearing at 8000-10000 r/min to obtain a mixture; then mixing the mixture with the streptomyces graminearus conidiospore powder, and grinding to obtain the streptomyces graminearus oil suspension.

Specifically, adding the above raw materials (except the streptomyces graminearus conidiospore powder) into a magnetic stirrer for premixing, and then shearing at high speed for 10-30 min by a Canadian homogenizer (800-1200 bar) or a high-speed shearing machine at 8000-10000 r/min, uniformly mixing to obtain the mixture; At room temperature, the mixture and the streptomyces graminearus conidiospore powder are added into a sand mill in proportion, grinding for 10-60 min to obtain the aforementioned streptomyces graminearus oil suspension.

The streptomyces graminearus oil suspension of the invention, which uses carrier oil as the dispersion medium, offers environmental friendliness, good permeability and adhesion properties, and rain washing resistance. Additionally, the carrier oil enhances the spore germination of streptomyces graminearus. Furthermore, it has the advantages of low application cost, slow spore settling speed, easy to shake well when using, stable efficacy, long storage time, suitability for various spraying techniques and a simple preparation method. The spore survival time of the streptomyces graminearus oil suspension is prelonged, with a spore germination rate is as high as 85% after 18 months of storage. It demonstrates effective control against yam, peanut and forest nematode, etc, examples include yam root knot nematode, pratylenchus coffeae, peanut root knot nematode, tomato meloidogyne incongnita, bursaphelenchus xylophilus, etc.

In another embodiment, the invention provides the application of the aforementioned streptomyces graminearus conidiospore powder, the aforementioned streptomyces graminearus oil suspension, or the aforementioned method for preparing the streptomyces graminearus oil suspension in preparing nematicides.

The following further describes the aforementioned streptomyces graminearus oil suspension and its preparation method with specific examples. This part further illustrates the content of the invention in combination with specific examples, but it should not be construed as a limitation of the invention. Unless otherwise specified, the technical means employed in the embodiment are conventional means well known to those skilled in the art. Unless otherwise specified, the reagents, methods and equipment used in the invention are conventional reagents, methods and equipment in the field. Unless otherwise specified, the reagents, methods and equipments used in this invention are conventional reagents, methods and equipments in this field.

### Example 1

The streptomyces graminearus oil suspension contains the following raw materials by mass fraction: 5% of the streptomyces graminearus conidiospore powder, 8% of SP-OF3472B, 0.5% of BHA, 0.5% of CMC-Na, 0.3% of calcium propionate, 3% of organic bentonite, 8% of wetting dispersant composed of Morwet EFW and NNO, 0.2% of xanthan gum, and 74.5% of rosin-based vegetable oil (OD-1 rosin-based vegetable oils produced by Fujian Nord Biotechnology Co., LTD, china), the mass ratio of the Morwet EFW to the NNO is 1:1.

The preparation method of the streptomyces graminearus conidiospore powder comprises the following steps:
S1. Inoculating the streptomyces graminearus strain onto a PDA medium plate, activating and culturing at 28° C. and 12L:12D until sporulation, and collecting spores powder from the PDA medium plate culture colony to prepare a suspension with a concentration of 3×10⁷ spores/mL;
S2. Adding 1 mL of the suspension to a PD culture medium to achieve a spore concentration of 1×10⁶ spores/mL, shaking culture at 28±1° C. and 180 r/min for 48 h, then transfering into 1000 mL of a PDA culture medium (PDA is standard fungal culture solution), and continuing shaking culture for 24 h, thus obtaining the inoculum;
S3. Placing 100 g of rice in a beaker, adding water according to the ratio of rice (g):water (mL)=1:1.5 (that is, adding 1.5 mL of water to each g of rice), soaking for 30 min, then washing and draining, and sterilizing for 30 min under the sterilization conditions of 121° C. and 0.1 MPa; After cooling, mixing the inoculum in step S2 with the rice according to the inoculation amount of 10% by volume, and the inoculated rice is poured into a Petri dish with a diameter of 15 cm and covered with plastic wrap; Culturing the inoculated at 28° C. and 12L:12D for sporulation, then removing the plastic wrap and culturing for 3-5 d to obtain a rice culture;
S4. After freezing and vacuum drying the rice culture, reducing the moisture rate to less than 4%, separating the spores powder from the rice substrate by vibrating screen, thereby obtaining the aforementioned streptomyces graminearus conidiospore powder. The spore content and viable spore rate were measured, the streptomyces graminearus conidiospore powder was stored in a refrigerator at 4° C. for later use.

The method for preparing the aforementioned streptomyces graminearus oil suspension comprises the following steps:
S1. Adding the raw materials other than the streptomyces graminearus conidiospore powder into a magnetic stirrer for premixing, shearing at 1500 r/min for 10 min to obtain a mixture; Then, placing the mixture into a Canadian homogenizer (working pressure is 800-1200 bar) for homogeneous mixing, so that the particle size of the mixture is controlled at about m, and the mixture is obtained after homogenization;
S2. At room temperature, the mixture and the streptomyces graminearus conidiospore powder are added into a sand mill in proportion, grinding for 20 min to obtain the aforementioned streptomyces graminearus oil suspension.

### Example 2

The streptomyces graminearus oil suspension of contains the following raw materials by mass fraction: 10% of the streptomyces graminearus conidiospore powder, 9% of DR7792, 0.5% of VC, 1.2% of BHA, 0.5% of calcium propionate, 5% of white carbon black, 12% of wetting dispersant composed of MF-5 (produced by Hubei Shangzhu Chemical Co., LTD, china) and NNO, 0.3% of corn dextrin, and 61.5% of sunflower seed oil, the mass ratio of the MF-5 to the NNO is 1:1.

Among them, the preparation method of the streptomyces graminearus conidiospore powder is same as EXAMPLE 1; The method for preparing the aforementioned streptomyces graminearus oil suspension is also same as EXAMPLE 1.

### Example 3

The streptomyces graminearus oil suspension contains the following raw materials by mass fraction: 15% of the streptomyces graminearus conidiospore powder, 17% of DR7792, 1.2% of VE, 2.5% of epoxidized soybean oil (Qilu Petrochemical), 1.2% of sodium diacetate, 7% of white carbon black, 15% of wetting dispersant composed of Morwet EFW and TERSPERSE 2020, 0.5% of nano zinc oxide, and 40.6% of corn oil, the mass ratio of the Morwet EFW to the TERSPERSE 2020 is 1:1.

Among them, the preparation method of the streptomyces graminearus conidiospore powder is same as EXAMPLE 1; The method for preparing the aforementioned streptomyces graminearus oil suspension is also same as EXAMPLE 1.

### Example 4

The streptomyces graminearus oil suspension contains the following raw materials by mass fraction: 20% of the streptomyces graminearus conidiospore powder, 18% of SP-OF3477 (produced by Nanjing Qingyu Chemical Research Co., Ltd., china), 2% of BHA, 1.5% of CMC-Na, 1.5% of sodium diacetate, 5% of organic bentonite, 15% of wetting dispersant composed of Morwet EFW and TERSPERSE 2020, 0.6% of nano zinc oxide, and 36.4% of sunflower seed oil, the mass ratio of the Morwet EFW to the TERSPERSE 2020 is 1:1.

Among them, the preparation method of the streptomyces graminearus conidiospore powder is same as EXAMPLE 1; The method for preparing the aforementioned streptomyces graminearus oil suspension is also same as EXAMPLE 1.

### Performance Test

### Effect of Additives on Spores and Preparations Performance

### (a) Screening of Additives

The tested additives (emulsifier, suspension stabilizer, antioxidant, preservative, thickener and wetting dispersant) were added in different proportions to an oil suspension temporarily prepared according to the spores powder: carrier oil=50 g: 1 L (w/v), and shook to disperse evenly to prepare the oil agent. Packed in penicillin bottles, sealed at room temperature and stored away from light. The spore germination rate was measured every 30 days.

Biocompatibility: Before sampling, the oil agent was homogenized on an oscillator, and then taking the above oil agent to prepare oil solutions with a concentration of 10⁶ spores/mL, after shaking culture at 28±1° C. and 180 r/min for 16 h, the spore germination rate (viable spore rate) was detected under microscope, and the influence of various additives was determined according to the spore germination rate. Each experiment was repeated 3 times, with spore water suspension as control. All spores with bud tube length equal to or greater than the spore diameter are regarded as germination.

### (b) Screening of Important Indicators of Preparations

The main indicators for evaluating the quality of the oil suspension include stability, suspension rate, redispersibility, flash point, viscosity, fluidity, etc. The most intuitive indicators are suspension rate and redispersibility.

Suspension rate: The property of the initial state of a solute dissolved or dispersed in a solvent in the oil suspension, which is related to fluidity, flash point, and viscosity.

Redispersibility: Refers to the property of the oil suspending that recover to its initial fully dispersed state after settling, which is related to fluidity, flash point, viscosity, etc.

The additives is selected based on three aspects: the biocompatibility of the additives, the influence of the additives on the suspension rate and redispersibility of the oil suspension.

### (c) Quality Testing of Preparations

The streptomyces graminearus oil suspension prepared in Example 1 has a viable spore rate of >80.00%, a moisture content ≤4.0, a pH value 5.6, an opening flash point >150° C., 98% passing the 25 μm test sieve, the viscosity (20° C.) of 0.42 Pa·s, and qualified thermal storage stability (30° C. for 2 months) and low-temperature stability.

### (3) Pharmacodynamic Test

Potting experiment: After disinfecting the surface of peanut seeds with 75% alcohol, peanut seeds were washed with sterile water for 3 times, then dried naturally, and the seeds were coated with 20 fold diluted liquid pesticide. Sterile water is used as a blank control. Sowing seeds in plastic pots filled with sterilized soil, with 3 seeds per pot, in preparation for pulling out seedlings with poor growth after budding, so as to ensure that there was one peanut seedling in each pot, and cultivate at room temperature under natural light. After four leaves have grown, each pot is inoculated 2 mL of second-instar larvae of meloidogyne hapla suspension with a concentration of 500 nematodes/mL. Each treatment is repeated for 3 times and arranged randomly. The seeds treated by sterile water were inoculated with the second-instar larvae of meloidogyne hapla suspension as control. After 50 days of inoculation, the number of oocysts was investigated. Calculating oocyst descent rate, and using the oocyst descent rate to represent the prevention and control effect:

$$Prevention and control effect ( \% ) = Oocyst descent rate ( \% ) = ( the number of oocysts in Control group - the number of oocysts in Treatment group ) / the number of oocysts in Control group \times 100 \% .$$

Evaluating the effect of promoting growth and increasing production based on the fresh weight of different treatments.

Field experiment: The experiment was carried out in the field of continuous cropping bergamot yam. The streptomyces graminearus oil suspension was diluted with water and root irrigation, while the blank control was root irrigation with clean water. Each treatment consisted of 3 communities and 3 repeats, each community covered an area of 10 square meters and arranged in random blocks. The liquid pesticide application adopts the method of diluting with water and irrigating roots, with a dosage of 300 mL per plant. The liquid pesticide was applied three times throughout the growth period, the first time was at one week after the yam seedlings emerge, and then every 20 days thereafter. After the yam harvest, investigating the growth promotion and increasing production of yam.

Table 3 below shows the classification criteria of tuber disease index.

TABLE 3
The classification criteria of tuber disease index

| Disease level | State of disease |
|---|---|
| 1 | A small amount of galls on the tuber (the area of galls |
| | accounts for less than 10.0% of the tuber area) |
| 3 | The area of galls accounts for |
| | 10.1% to 25.0% of the tuber area |
| 5 | The area of galls accounts for |
| | 25.1% to 50.0% of the tuber area |
| 7 | The area of galls accounts for |
| | 50.1% to 75.0% of the tuber area |
| 9 | The area of galls accounts for greater |
| | than 75.1% of the tuber area |

The disease index (DI) was calculated according to the following formula.

$$DI = 100 \times Σ ( N i \times i ) / ( Nt \times 9 ) ;$$

Where: DI is the disease index, Ni is the number of plants at each disease level, i is the corresponding disease level value, and Nt is the total number of investigated plants.

(4) Pharmacodynamic Test Against Bursaphelenchus xylophilus

When most nematodes grown on Botrytis cinerea culture medium were in the pregnancy stage (J4/adult), the nematodes were homogenized at the same age (growth period) to ensure that all nematodes were at the same growth level. The nematodes were collected in a centrifuge tube after standing for 12 h using the Belman funnel method, then they were centrifuged at 3500 r/min for 4 min, rinsed with 0.9% of normal saline for 3 times to prepare a nematode suspension of 100 nematodes per milliliter. 0.5 mL of the nematode suspension were added to each hole of the 24 well plates, followed by an equal volume of diluted liquid pesticide, with the background solution as a blank control. Three parallel experiments were set up to observe the survival status of the nematodes under a stereoscopic microscope at 36, 48 and 72 hours after treatment, respectively. The nematodes is judged to be alive when it moves in a spiral shape, and dead when it does not move and its body is stiff. The mortality rate and the corrected mortality rate was calculated according to the following formula.

$$Mortality rate = ( the number of dead nematodes / the total number of tested insects ) \times 100 \% ;$$

$$Corrected mortality rate = [ ( the number of dead nematodes in Treatment group - the number of dead nematodes in Control group ) / ( 1 - the number of dead nematodes in Control group ) ] \times 100 \% .$$

The Potting experiment results of the streptomyces graminearus oil suspension prepared in Example 1 are shown in Table 4 below.

TABLE 4
The Potting experiment results of the streptomyces graminearus oil
suspension prepared in Example 1 against peanut root knot nematode

| | Treatment | Disease | Prevention and | Increasing |
|---|---|---|---|---|
| Preparations | concentration | index | control effect | production (%) |
| 5% of the streptomyces graminearus | 200-fold dilution | 25.32 | 71.26 | 25.31 |
| oil suspension prepared in Example 1 | | | | |
| 5% of abamectin cream | 1000-fold dilution | 33.76 | 58.47 | 8.12 |
| 200 million spores per gram of | 100-fold dilution | 41.25 | 47.21 | 10.15 |
| Purpureocillium lilacinum | | | | |
| Clean water blank control | — | 82.37 | — | |

According to Table 4, the streptomyces graminearus oil suspension prepared in Example 1 had a control effect of over 70% against peanut root knot nematode; However, the control pesticides abamectin and Purpureocillium lilacinum was only 58.47% and 47.21%, respectively. In terms of increasing production and promoting growth, 5% of the streptomyces graminearus oil suspension increased production by more than 25%, and other control agents also had a certain increase in production effect, around 10%.

The Field experiment results of the streptomyces graminearus oil suspension prepared in Example 4 are shown in Table 5 below.

TABLE 5
The Potting experiment results of the streptomyces graminearus
oil suspension prepared in Example 4 against yam root knot nematode

| | Treatment | Disease | Prevention and | Increasing |
|---|---|---|---|---|
| Preparations | concentration | index | control effect | production (%) |
| 20% of the streptomyces graminearus | 1000-fold dilution | 21.30 | 52.14 | 11.56 |
| oil suspension prepared in Example 4 | | | | |
| 20% of the streptomyces graminearus | 500-fold dilution | 13.15 | 78.22 | 19.12 |
| oil suspension prepared in Example 4 | | | | |
| 20% of the streptomyces graminearus | 200-fold dilution | 8.02 | 83.88 | 22.36 |
| oil suspension prepared in Example 4 | | | | |
| Clean water blank control | — | 49.11 | — | |

According to Table 5, it can be seen that the control effect of the three concentrations of the streptomyces graminearus oil suspension prepared in Example 4 on yam root-knot nematodes was significantly different. The control effect sequence was 200 fold dilution, 500 fold dilution and 1000 fold dilution of the streptomyces graminearus oil suspension. The three treatment concentrations of the streptomyces graminearus oil suspension showed a significant effects on promoting growth and increasing production, among which the 200 fold dilution treatment resulted in a production increase of 22.36%.

The nematicidal activity of the streptomyces graminearus oil suspension prepared in Example 4 was tested, and the nematicidal activity of the streptomyces graminearus oil suspension prepared in Example 4 at 500 fold dilution against Bursaphelenchus xylophilus is shown in Table 6 below.

TABLE 6
The nematicidal activity of the streptomyces graminearus oil suspension
at 500 fold dilution against Bursaphelenchus xylophilus

| Time (hours) | Corrected mortality rate /% |
|---|---|
| 36 | 32.51 ± 1.36 |
| 48 | 66.33 ± 2.56 |
| 72 | 100 ± 0.00 |

According to Table 6, it can be seen that the control effect of the streptomyces graminearus oil suspension prepared in Example 4 at 500 fold dilution on Bursaphelenchus xylophilus gradually increased over time, the mortality rate of Bursaphelenchus xylophilus was increased gradually. After 72 hours, the corrected mortality rate of Bursaphelenchus xylophilus at 500 fold dilution of the 20% streptomyces graminearus oil suspension can reach as high as 100%.

The storage stability of the streptomyces graminearus oil suspension prepared in Example 1 at room temperature (25° C.) was tested, and the results are shown in Table 7 below.

TABLE 7
The storage stability of the streptomyces graminearus
oil suspension at room temperature

| | Storage time at room temperature (month) | | | | |
|---|---|---|---|---|---|
| | 0 | 3 | 6 | 18 | 24 |
| Viable spore rate (%) | 97.00 | 91.29 | 88.10 | 85.64 | 70.91 |

As can be seen from Table 7, the spore survival time of the streptomyces graminearus oil suspension is long, and the spore germination rate is as high as 85% after 18 months of storage.

The above is only the preferred embodiment of the invention, and it is not used to limit the invention. Any modification, equivalent substitution, improvement, etc. made within the spirit and principle of the invention should be included in the protection scope of the invention.

What is claimed is:

1. A streptomyces graminearus oil suspension, containing the following raw materials by mass fraction: 3-25% of streptomyces graminearus conidiospore powder, 5-10% of emulsifier, 0.5-3% of stabilizer, 3-8% of wetting dispersant, 0.1-3% of antioxidant, 3-8% of thickener, 0.1-0.5% of ultraviolet protectant, 0.1-3% of preservative and 39.5-85.2% of carrier oil;
wherein the streptomyces graminearus conidiospore powder is prepared by activating and culturing a streptomyces graminearus strain to produce spores, thereby obtaining the streptomyces graminearus conidiospore powder; the streptomyces graminearus strain has a deposit accession number of CCTCC NO: M 2022512.

2. The streptomyces graminearus oil suspension of claim 1, wherein a preparation method of the streptomyces graminearus conidiospore powder comprises:
inoculating the streptomyces graminearus strain onto a PDA medium plate, activating and culturing until sporulation, and collecting spores powder from the PDA medium plate culture colony to prepare a suspension with a concentration of (2-4)×10⁷ spores/mL;
adding 0.5-2 mL of the suspension to a PD culture medium to achieve a spore concentration of (0.5-2)×10⁶ spores/mL, shaking culture, then transfering into a PDA culture medium, and continuing shaking culture to obtain an inoculum;
adding rice to the inoculum, and culturing for sporulation to obtain a rice culture;
after freezing and vacuum drying the rice culture, separating the spores powder from the rice substrate, thereby obtaining the streptomyces graminearus conidiospore powder.

3. The streptomyces graminearus oil suspension of claim 1, wherein the carrier oil comprises at least one of rosin-based vegetable oil, soybean oil, sunflower seed oil, and corn oil;
wherein the emulsifier comprises at least one of SP-OF3472B, DR7792, SP-OF3477, TERMUL2500, TERMUL3015, LT-F192, and op-10;
wherein the stabilizer comprises at least one of CMC-Na, BHA, and epoxidized soybean oil;
wherein the wetting dispersant comprises at least one of Morwet EFW, TERWET 1010, MF-5, Morwet D425, calcium lignosulphonate, TERSPERSE 2020, and NNO;
wherein the antioxidant comprises at least one of vitamin C, vitamin E, and BHA;
wherein the thickener comprises at least one of silica and organic bentonite;
wherein the ultraviolet protectant comprises at least one of dextrin, xanthan gum, sodium fluorescein, vitamin C, and nano zinc oxide;
wherein the preservative comprises at least one of sodium diacetate and calcium propionate.

4. The streptomyces graminearus oil suspension of claim 1, wherein the streptomyces graminearus conidiospore powder has a spore content greater or equal to 7×10⁹ spores/g, and a moisture content of 2-5% by weight.

5. A method for preparing the streptomyces graminearus oil suspension, said method comprising:
mixing streptomyces graminearus conidiospore powder, emulsifier, stabilizer, wetting dispersant, antioxidant, thickener, ultraviolet protectant, preservative, and carrier oil, and stirring to obtain the streptomyces graminearus oil suspension;
wherein the streptomyces graminearus oil suspension contains the following raw materials by mass fraction: 3-25% of the streptomyces graminearus conidiospore powder, 5-10% of the emulsifier, 0.5-3% of the stabilizer, 3-8% of the wetting dispersant, 0.1-3% of the antioxidant, 3-8% of the thickener, 0.1-0.5% of the ultraviolet protectant, 0.1-3% of the preservative and 39.5-85.2% of the carrier oil;
wherein the streptomyces graminearus conidiospore powder is prepared by activating and culturing a streptomyces graminearus strain to produce spores, thereby obtaining the streptomyces graminearus conidiospore powder; the streptomyces graminearus strain has a deposit accession number of CCTCC NO: M 2022512.

6. The method of claim 5, said method comprising:
mixing the emulsifier, the stabilizer, the wetting dispersant, the antioxidant, the thickener, the ultraviolet protectant, the preservative, and the carrier oil, and shearing at 8000-10000 r/min to obtain a mixture;
mixing the mixture with the streptomyces graminearus conidiospore powder, and grinding to obtain the streptomyces graminearus oil suspension.

7. The method of claim 5, wherein the streptomyces graminearus conidiospore powder is prepared by the following steps:
inoculating the streptomyces graminearus strain onto a PDA medium plate, activating and culturing until sporulation, and collecting spores powder from the PDA medium plate culture colony to prepare a suspension with a concentration of (2-4)×10⁷ spores/mL;
adding 0.5-2 mL of the suspension to a PD culture medium to achieve a spore concentration of (0.5-2)×10⁶ spores/mL, shaking culture, then transfering into a PDA culture medium, and continuing shaking culture to obtain an inoculum;
adding rice to the inoculum, and culturing for sporulation to obtain a rice culture;
after freezing and vacuum drying the rice culture, separating the spores powder from the rice substrate, thereby obtaining the streptomyces graminearus conidiospore powder.

8. The method of claim 5, wherein the carrier oil comprises at least one of rosin-based vegetable oil, soybean oil, sunflower seed oil, and corn oil;
wherein the emulsifier comprises at least one of SP-OF3472B, DR7792, SP-OF3477, TERMUL2500, TERMUL3015, LT-F192, and op-10;
wherein the stabilizer comprises at least one of CMC-Na, BHA, and epoxidized soybean oil;
wherein the wetting dispersant comprises at least one of Morwet EFW, TERWET 1010, MF-5, Morwet D425, calcium lignosulphonate, TERSPERSE 2020, and NNO;
wherein the antioxidant comprises at least one of vitamin C, vitamin E, and BHA;
wherein the thickener comprises at least one of silica and organic bentonite;
wherein the ultraviolet protectant comprises at least one of dextrin, xanthan gum, sodium fluorescein, vitamin C, and nano zinc oxide;
wherein the preservative comprises at least one of sodium diacetate and calcium propionate.

9. The method of claim 5, wherein the streptomyces graminearus conidiospore powder has a spore content greater or equal to 7×10⁹ spores/g, and a moisture content of 2-5% by weight.

10. Application of the streptomyces graminearus oil suspension of claim 1 in preparing nematicides.


SEQUENCE LISTING (1 sequence)

SEQ ID NO: 1 - DNA, 1414 bp, Streptomyces graminearus
  source 1..1414: mol_type = genomic DNA
catgcaagtcgaacgatgaagcccttcggggtggattagtggcgaacgggtgagtaacacgtgggcaatctgccctgcactctgggacaagccctggaaacggggtctaataccggatatgaccatcttgggcatccttgatggtgtaaagctccggcggtgcaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgattgtgaaagctcggggcttaaccccgagtctgcagtcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtacaggtggtgcatggctgtcgtcagctcgtgtcgtgagatgttgggttaagtcccgcaacgagcgcaacccttgtcccgtgttgccagcaggcccttgtggtgctggggactcacgggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggaccagcgat